(19) United States
(12) Patent Application Publication
(10) Pub. No.: US 2015/0119615 A1
(43) Pub. Date: Apr. 30, 2015

(54) PYROLYSIS GASOLINE TREATMENT PROCESS

(71) Applicant: UOP LLC, Des Plaines, IL (US)

(72) Inventors: Robert J. Schmidt, Barrington, IL (US); Charles P. Luebke, Mount Prospect, IL (US); Rose M. Janulis, Oak Brook, IL (US)

(73) Assignee: UOP LLC, Des Plaines, IL (US)

(21) Appl. No.: 14/063,542

(22) Filed: Oct. 25, 2013

(51) Int. Cl. C07C 5/05

(52) U.S. Cl. CPC C07C 5/05
USPC 585/259

(57) ABSTRACT

A process for treating pyrolysis gasoline that includes providing a first stage di-olefin reactor that includes a first bed and a second bed and introducing a pyrolysis gasoline stream to the first bed of the first stage di-olefin reactor. The process also preferably includes providing interstage cooling to the pyrolysis gasoline stream between the first and second beds of the first stage di-olefin reactor and routing the cooled pyrolysis gasoline stream through the second bed of the first stage di-olefin reactor. Finally, embodiments of the process also preferably involve routing at least a portion of an effluent stream from the second bed of the first stage di-olefin reactor to a location upstream of the first bed of the first stage di-olefin reactor, such that the effluent stream is configured to be combined with the pyrolysis gasoline stream.

The present invention relates generally to processes for treating pyrolysis gasoline, and more specifically to processes for treating pyrolysis gasoline to remove dienes and olefins prior to downstream processing to remove benzene, toluene and xylene isomers (commonly referred to as BTX processing).

### BACKGROUND OF THE INVENTION

The treatment of pyrolysis gasoline to remove dienes and olefins prior to downstream BTX processing for high value para-xylene (PX) remains a challenge. Currently, the process requires two steps and the high heat of reaction needed for these steps requires high effluent recycle rates to maintain the resulting temperature rise at an acceptable delta temperature performance. The key steps include: (1) a first stage to saturate di-olefins; and (2) a second stage to hydrotreat the remaining olefins and aromatics to remove sulfur and nitrogen species down to a level of less than 0.5 ppm to make the net product stream acceptable for further processing in a downstream aromatics complex for high value PX production. The current technology is limited in that heat control in the first and second stages requires high selectivity catalysts to be used in the lead stage, followed by careful heat management in the second stage to reduce recycle rates to minimize utilities consumption and capital costs.

### BRIEF SUMMARY OF THE INVENTION

Achieving very high di-olefin (DO) selectivity in the first stage of a two stage pyrolysis gasoline hydrotreating unit is essential in achieving low polymerization of feed di-olefins and long catalyst life. The process described herein relates to a two reactor scheme with inter-stage cooling for the first stage that keeps the average catalyst bed temperature very low, such as from 70° C. to 90° C. at the start-of-run (SOR) and from 110° C. to 130° C. at the end-of-run (EOR). This scheme results in improved DO to olefin selectivity at high conversion, and an all liquid phase condition at the second stage reactor outlet. Such a process coupled with a vapor phase split feed to a two bed reactor scheme for the second hydrotreating stage results in improved yields of benzene, toluene and xylene isomers (commonly referred to as BTX) and minimum deactivation across both stages.

More specifically, certain embodiments of the present invention relate to a process for treating pyrolysis gasoline that includes providing a first stage di-olefin reactor that includes a first bed and a second bed, and introducing a pyrolysis gasoline stream to the first bed of the first stage di-olefin reactor. The process also preferably includes providing interstage cooling to the pyrolysis gasoline stream between the first and second beds of the first stage di-olefin reactor, and routing the cooled pyrolysis gasoline stream through the second bed of the first stage di-olefin reactor. Finally, embodiments of the process also preferably involve routing at least a portion of an effluent stream from the second bed of the first stage di-olefin reactor to a location upstream of the first bed of the first stage di-olefin reactor, such that the effluent stream is configured to be combined with the pyrolysis gasoline stream.

Certain embodiments of the present process also relate to a process for treating pyrolysis gasoline that includes providing a first stage di-olefin reactor that includes a first bed and a second bed, and introducing a pyrolysis gasoline stream to the first bed of the first stage di-olefin reactor. Such embodiments also preferably include providing interstage cooling to the pyrolysis gasoline stream between the first and second beds of the first stage di-olefin reactor, and then routing the cooled pyrolysis gasoline stream through the second bed of the first stage di-olefin reactor. Next, such embodiments could also include performing a fractionation process on at least a portion of the effluent stream from the second bed of the first stage di-olefin reactor.

Additionally, embodiments of the present process also relate to a process for treating pyrolysis gasoline that includes providing a first stage di-olefin reactor that includes a first bed and a second bed, as well as providing a second stage hydrotreating reactor. The process of such embodiments also preferably involves introducing a pyrolysis gasoline stream to the first bed of the first stage di-olefin reactor, and providing interstage cooling to the pyrolysis gasoline stream between the first and second beds of the first stage di-olefin reactor. Such embodiments also preferably include routing the cooled pyrolysis gasoline stream through the second bed of the first stage di-olefin reactor, and performing a fractionation process on at least a portion of an effluent stream from the second bed of the first stage di-olefin reactor. After fractionating, a vapor phase resultant stream from the fractionation process is routed to the second stage hydrotreating reactor. Finally, the process of such embodiments preferably includes performing hydrotreating within the second stage hydrotreating reactor such that a liquid phase effluent stream results, without any gas phase effluent stream.

### BRIEF DESCRIPTION OF THE DRAWING

A preferred embodiment of the present invention is described herein with reference to the drawing wherein:

FIG. 1 is an example of an embodiment of the present process for treating pyrolysis gasoline; and

FIG. 2 is a schematic of one example of a first stage reactor that can be used in the process of FIG. 1.

### DETAILED DESCRIPTION OF THE INVENTION

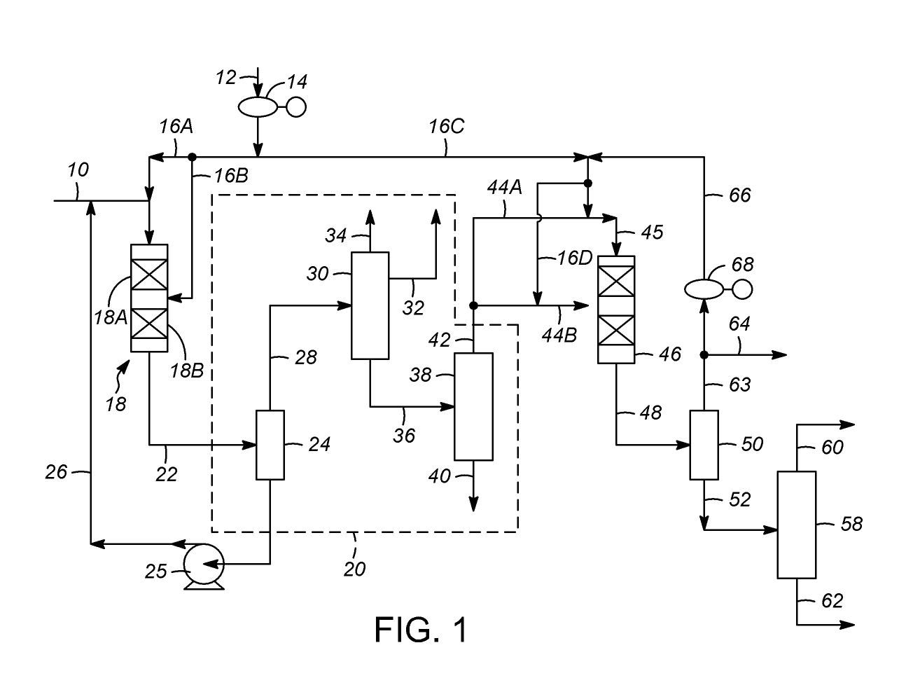

An example of an embodiment of the present process will now be described. More specifically, FIG. 1 is a process flow diagram that shows one example of a process for treating pyrolysis gasoline. Of course, other embodiments are also contemplated, as well as modifications to the FIG. 1 embodiment. For example, another similar embodiment of a process flow diagram is disclosed in application Ser. No. ______ [GBC Docket No. 5066.115487; UOP No. H00-37937-8200], which is assigned to the same Assignee as the present application, and which is hereby incorporated by reference it its entirety into the present application. Also, FIG. 1 is merely a schematic of the process flow, and therefore various features (such as processors, controllers, valves, sensors, etc.) are not shown. However, such additional features are known to those of ordinary skill in the art, and therefore are not necessary for an understanding or implementation of the present process.

The feed stream 10 of FIG. 1 is a pyrolysis gasoline stream that preferably contains a full range of C5 to C10 hydrocarbons. Preferably, the pyrolysis gasoline stream 10 is in the liquid phase, and is at a temperature within the range of approximately 40° C. to approximately 60° C. in the inlet to the first stage catalyst, and a pressure within the range of approximately 350 to approximately 850 psig, or at a pressure that is at least sufficient to maintain substantially all of the hydrocarbons in the liquid phase. In this embodiment, a make-up hydrogen stream 12 is introduced into a make-up hydrogen compressor 14 prior to being split into a first make-up hydrogen stream 16A, a second make-up hydrogen stream 16B, and a third make-up hydrogen stream 16C. The make-up hydrogen streams 16A, 16B and 16C are controlled according to any desired method to provide the necessary make-up hydrogen to the associated stream, such as the pyrolysis gasoline stream 10. Although the make-up hydrogen streams 16A, 16B and 16C are in the vapor phase, they are being combined in such low percentages (for example about 2-3%) with the liquid phase streams (such as the pyrolysis gasoline stream 10 or stream 44A), that the gas phase hydrogen quickly dissolves, and the resulting combined stream remains in liquid phase.

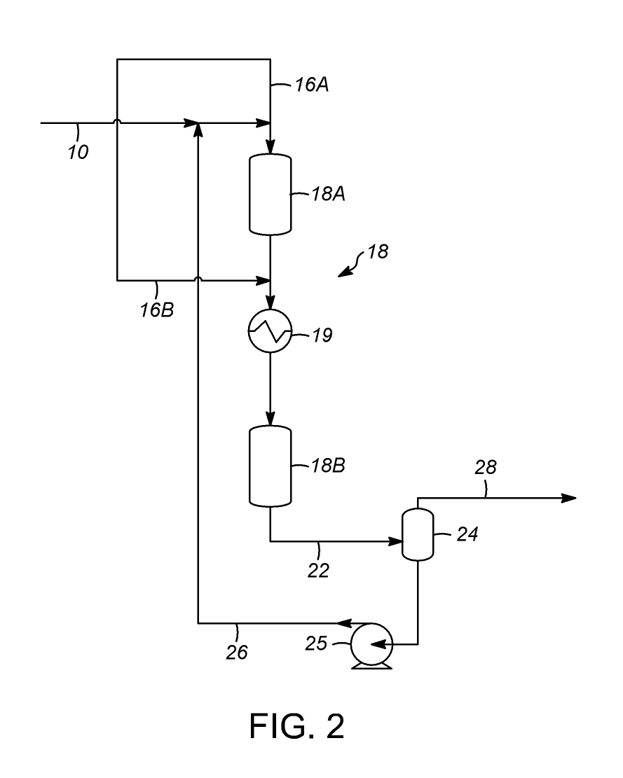

After receiving make-up hydrogen from the make-up hydrogen stream 16A, if necessary, the pyrolysis gasoline stream 10 is directed to a first stage reactor 18, which in this embodiment is preferably a di-olefin reactor that is used for removing di-olefins from the pyrolysis gasoline with a catalyst. Although other types of reactors are contemplated for the first stage reactor 18, one example of a preferred reactor is a two bed reactor with a cooler between the beds, such as represented by reactor 18 of FIG. 2. More specifically, the first stage di-olefin reactor 18 preferably includes a first catalyst bed 18A and a second catalyst bed 18B, with an intercooler 19 between the beds. The intercooler 19 may comprise any desired cooling mechanism, such as a heat exchanger. Further, the intercooler 19 may be housed within the vessel of the reactor 19, or cooling may be provided by a mechanism outside of the reactor vessel itself.

Preferably, the catalyst used in both beds of the di-olefin reactor 18 is preferably a high selectivity di-olefin saturation catalyst. For example, a high selectivity di-olefin saturation catalyst consisting of a shell impregnated palladium (Pd) system or a Pd layered sphere could be used. Alternatively, the catalyst could include engineered catalyst support (ECS). One example of a catalyst that could be used is an eggshell catalyst with approximately 100 microns of Pd loaded onto an outer layer at between 0.1 and 0.4 wt % Pd overall on a theta aluminum spherical support ranging from 60 to 90 m²/gm SA. Sufficient performance could also be obtained with a conventional PF-4 catalyst, which is a spherical R-9 catalyst with 0.4% Pd, 0.5% Li that has been reduced and cold sulfided, although catalysts with an eggshell Pd profile are preferred for certain embodiments. It is contemplated that either the same catalyst is used in both bed 18A and bed 18B, or that different catalysts, or different formulations of the same catalyst, are used in beds 18A and 18B.

As can be seen in FIG. 2, in addition to routing the first hydrogen stream 16A to the first bed 18A of the first stage di-olefin reactor 18, as discussed above, the present process preferably also includes routing the second hydrogen stream 16B to the second bed 18B of the first stage di-olefin reactor 18, without having the second hydrogen stream 16B pass through the first bed 18A of the first stage di-olefin reactor 18. Preferably, the routing of the second hydrogen stream 16B to the second bed 18B of the first stage di-olefin reactor 18 is performed upstream of the step of providing interstage cooling via intercooler 19 to the pyrolysis gasoline stream between the first bed 18A and the second bed 18B of the first stage di-olefin reactor 18.

After the pyrolysis gasoline has been routed through both beds of the first stage reactor 18, a fractionation process can be performed upon the pyrolysis gasoline stream. Dashed box 20 of FIG. 1 contains one example of a fractionation process that can be used to separate the C5 and the C10+ hydrocarbons from the stream, but of course other configurations of components and processes for fractionation are also contemplated. In the fractionation process 20, stream 22 is routed to a first stage surge drum 24. A resultant liquid stream 26 from the surge drum 24 is routed, via recycle pump 25, as a recycle stream that is combined with the pyrolysis gasoline stream 10 at a location upstream of the first bed 18A of the first stage reactor 18.

Another resultant stream 28 from the surge drum 24, which stream is preferably in a vapor phase, is routed to a depentanizer column 30 (FIG. 1), or other similar component, for removing pentane and lighter fractions from the pyrolysis gasoline stream. After processing within the depentanizer column 30, the removed C5 hydrocarbons will be in stream 32, which stream can be further processed if desired, and a vent gas stream 34 will also result. Further, the processed pyrolysis gasoline, which now lacks the C5 hydrocarbons, is routed via stream 36 to a rerun column 38 for the removal of the C10+ hydrocarbons, which exit column 38 via stream 40. Stream 40 can be further processed, as desired. As an alternative, the C9 hydrocarbons can also be removed, if desired, such that resultant stream 42 is a pyrolysis gasoline stream containing C6 to C8 hydrocarbons.

The resultant stream 42 from the rerun column 38, which in this embodiment is a pyrolysis gasoline stream containing C6 to C9 hydrocarbons (as the C5 and C10+ hydrocarbons have been removed during the fractionation process 20), is then split into a first stream 44A and a second stream 44B. Preferably, streams 44A and 44B are both liquid phase streams. These streams 44A and 44B are then preferably vaporized in a heater/exchanger (not shown), and then combined with hydrogen to ensure that an all vapor phase condition exists in the inlet to the catalyst bed. This ensures that good flow distribution is maintained in the all vapor phase reaction without the need for special distributor nozzles or plates to handle a mixed phase feed condition.

Both stream 44A and stream 44B are routed to a second stage reactor 46, which in this embodiment is preferably a hydrotreater reactor with two catalyst beds (such as an upper bed in a first portion of the reactor and a lower bed in a second portion of the reactor). In certain embodiments, the catalyst(s) and process parameters of reactor 46 are selected such that the remaining olefins and aromatics are selectively saturated, and the sulfur and nitrogen species are hydrotreated without their aromatics being saturated. The same catalyst may be used in both portions of the second stage reactor 46, or different catalysts or different formulations of the same catalyst, could be used in each portion. Further, a mix of two, or more, different catalysts could be used in each portion of reactor 46, whereby either the same ratio of components of the catalyst are used in both portions of reactor 46, or different ratios of the same components are used in each of the two portions of reactor 46. Finally, it also contemplated that a reactor with more than two beds, and/or with more than two feeds, could also be used as reactor 46.

In one exemplary embodiment, the catalyst in both the first and second portions of second stage reactor 46 comprises a catalyst that is a combination of a Ni—Mo catalyst and a Co—Mo catalyst, where there is between 20-30% of the Ni—Mo component and between 70-80% of the Co—Mo component. As mentioned above, the catalyst for the first and second portions could be the same (such as a 30/70% split for Ni—Mo/Co—Mo) or two different formulations could be used (such as a 30/70% of Ni—Mo/Co—Mo for the first portion and a 20/80% split of Ni—Mo/Co—Mo for the second portion, or vice-versa).

Preferably, the third make-up hydrogen stream 16C (mentioned above) is configured to be combined with stream 44A prior to the combined stream 45 entering the first portion of the second stage reactor 46. The amount of make-up hydrogen needed can be determined and controlled in any desired manner. Preferably, a fourth make-up hydrogen stream 16D, which can be split from third hydrogen stream 16C, or can originate at another point in the process, is configured to be combined with stream 44B prior to the combined stream entering the second portion of the second stage reactor 46. Once again, the amount of make-up hydrogen needed can be determined and controlled in any desired manner.

In the FIG. 1 embodiment, the effluent stream 48 from the second stage reactor 46 is routed to a separator 50. Preferably, the hydrotreating reaction within the second stage reactor is performed under such conditions that all effluent (i.e. stream 48) is in the liquid phase, without any gas phase effluent stream. Also, in preferred embodiments, there is no liquid recycle stream from separator 50 to the second stage reactor 46 because the recycle gas stream 63/66 (described below) will provide sufficient cooling for many applications. However, it is contemplated that the liquid phase effluent stream 52 from the separator 50 can be split, if desired, into a recycle stream (not shown) that can be configured to be combined with stream 45 upstream of the first portion of the second stage reactor 46.

Turning back to the process at the point of the separator 50, the stream 52 from the separator 50 is preferably routed to a stripper, such as debutanizer 58, where it is processed to form a stream 60, which contains the C4 hydrocarbons, and a stream 62, which contains the C6 to C8 hydrocarbons. Preferably, the stream 62 is a liquid phase stream and the stream 60 is a vapor phase stream

In addition to the liquid phase effluent stream 52, a gas phase effluent stream 63 is also created by the separator 50. This gas phase effluent stream 63 is split so that it can either be routed off as vent gas via stream 64, or it can be used as recycle gas via recycle gas stream 66. As can be seen in FIG. 1, the recycle gas stream 66 passes through a recycle gas compressor 68 prior to being combined with streams 44A and 16C to form combined stream 45, which is routed into the first portion of the second stage compressor 46.

The embodiments of the process described herein provide at least some of the following highlights/advantages:

(1) The first stage reactor preferably includes two catalyst beds with interstage cooling.

(2) All fresh feed and recycle are preferably provided to the first bed of the first stage reactor.

(3) Hydrogen is preferably split between the first and second beds of the first stage reactor.

(4) A cooler is preferably provided between the beds of the first stage reactor, thereby keeping the reactor outlet temperature at a minimum for increased di-olefin to olefin selectivity.

(5) The reactor effluent is preferably recycled for temperature rise control.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention. It is understood that various changes may be made in the function and arrangement of elements described in an exemplary embodiment without departing from the scope of the invention as set forth in the appended claims.

What is claimed is:

1. A process for treating pyrolysis gasoline comprising:
providing a first stage di-olefin reactor that includes a first bed and a second bed;
introducing a pyrolysis gasoline stream to the first bed of the first stage di-olefin reactor;
providing interstage cooling to the pyrolysis gasoline stream between the first and second beds of the first stage di-olefin reactor;
routing the cooled pyrolysis gasoline stream through the second bed of the first stage di-olefin reactor; and
routing at least a portion of an effluent stream from the second bed of the first stage di-olefin reactor to a location upstream of the first bed of the first stage di-olefin reactor, such that the effluent stream is configured to be combined with the pyrolysis gasoline stream.

2. The process according to claim 1, further comprising;
splitting a hydrogen stream into a first hydrogen stream and a second hydrogen stream;
routing the first hydrogen stream to the first bed of the first stage di-olefin reactor; and
routing the second hydrogen stream to the second bed of the first stage di-olefin reactor, without having the second hydrogen stream pass through the first bed of the first stage di-olefin reactor.

3. The process according to claim 2, wherein the routing of the second hydrogen stream to the second bed of the first stage di-olefin reactor is performed upstream of the step of providing interstage cooling to the pyrolysis gasoline stream between the first and second beds of the first stage di-olefin reactor.

4. The process according to claim 1, further comprising:
performing a fractionation process on at least a portion of the effluent stream from the second bed of the first stage di-olefin reactor;
after performing the fractionation process, splitting a resultant stream into a first resultant stream and a second resultant stream; and
routing the first resultant stream to a first portion of a second stage hydrotreating reactor and routing the second resultant stream to a second portion of the second stage hydrotreating reactor.

5. The process according to claim 4, wherein the fractionation process comprises the following steps that are performed prior to the step of splitting the resulting steam into a first resultant stream and a second resultant stream:
routing the portion of the effluent stream upon which the fractionation process is being performed through a depentanizer column; and
routing a resultant liquid stream from the depentanizer column to a rerun column.

6. The process according to claim 4, wherein the fractionation process comprises the following steps that are performed prior to the step of splitting the resulting steam into a first resultant stream and a second resultant stream:
routing the portion of the effluent stream upon which the fractionation process is being performed to a first stage effluent drum;
routing a resultant stream from the first stage effluent drum to a depentanizer column; and
routing a resultant liquid stream from the depentanizer column to a rerun column.

7. The process according to claim 4, further comprising:
routing a liquid phase effluent steam from the second stage hydrotreating reactor to a separator;
routing a liquid phase effluent stream from the separator to a stripper; and
obtaining a resultant stream including C6 to C9 hydrocarbons from the stripper.

8. A process for treating pyrolysis gasoline comprising:
providing a first stage di-olefin reactor that includes a first bed and a second bed;
introducing a pyrolysis gasoline stream to the first bed of the first stage di-olefin reactor;
providing interstage cooling to the pyrolysis gasoline stream between the first and second beds of the first stage di-olefin reactor;
routing the cooled pyrolysis gasoline stream through the second bed of the first stage di-olefin reactor; and
performing a fractionation process on at least a portion of the effluent stream from the second bed of the first stage di-olefin reactor.

9. The process according to claim 8, further comprising:
after performing the fractionation process, routing a resultant stream to a second stage hydrotreating reactor.

10. The process according to claim 8, further comprising:
after performing the fractionation process, splitting a resultant stream into a first resultant stream and a second resultant stream; and
routing the first resultant stream to a first portion of a second stage hydrotreating reactor and routing the second resultant stream to a second portion of the second stage hydrotreating reactor.

11. The process according to claim 8, further comprising:
after performing the fractionation process, splitting a vapor phase resultant stream into a first vapor phase resultant stream and a second vapor phase resultant stream; and
routing the first vapor phase resultant stream to a first portion of a second stage hydrotreating reactor and routing the second vapor phase resultant stream to a second portion of the second stage hydrotreating reactor.

12. The process according to claim 11, further comprising:
performing a reaction within the second stage hydrotreating reactor under conditions such that all effluent from the second stage hydrotreating reactor is in a liquid phase.

13. The process according to claim 9, further comprising:
routing a liquid phase effluent stream from the second stage hydrotreating reactor to a separator;
routing a liquid phase effluent stream from the separator to a stripper; and
obtaining a resultant stream including C6 to C9 hydrocarbons from the stripper.

14. A process for treating pyrolysis gasoline comprising:
providing a first stage di-olefin reactor that includes a first bed and a second bed;
providing a second stage hydrotreating reactor;
introducing a pyrolysis gasoline stream to the first bed of the first stage di-olefin reactor;
providing interstage cooling to the pyrolysis gasoline stream between the first and second beds of the first stage di-olefin reactor;
routing the cooled pyrolysis gasoline stream through the second bed of the first stage di-olefin reactor;
performing a fractionation process on at least a portion of an effluent stream from the second bed of the first stage di-olefin reactor;
obtaining a vapor phase resultant stream from the fractionation process;
routing the vapor phase resultant stream to the second stage hydrotreating reactor; and
performing hydrotreating within the second stage hydrotreating reactor such that a liquid phase effluent stream results, without any gas phase effluent stream.

15. The process according to claim 14, wherein the second stage hydrotreating reactor includes a first bed and a second bed.

16. The process according to claim 15, further comprising:
after performing the fractionation process, splitting a vapor phase resultant stream into a first vapor phase resultant stream and a second vapor phase resultant stream; and
routing the first vapor phase resultant stream to a first portion of the second stage hydrotreating reactor and routing the second vapor phase resultant stream to a second portion of the second stage hydrotreating reactor.

17. The process according to claim 16, wherein the fractionation process comprises the following steps that are performed prior to the step of splitting the resulting steam into a first vapor phase resultant stream and a second vapor phase resultant stream:
routing the portion of the effluent stream upon which the fractionation process is being performed to a first stage effluent drum;
routing a resultant stream from the first stage effluent drum to a depentanizer column; and
routing a resultant liquid stream from the depentanizer column to a rerun column.

18. The process according to claim 14, further comprising:
routing a liquid phase effluent steam from the second stage hydrotreating reactor to a separator;
routing a liquid phase effluent stream from the separator to a stripper; and
obtaining a resultant stream including C6 to C9 hydrocarbons from the stripper.
